(19) United States
(12) Patent Application Publication
(10) Pub. No.: US 2026/0102194 A1
(43) Pub. Date: Apr. 16, 2026

(54) MEDICAL DEVICES, SYSTEMS, AND RELATED METHODS FOR STIMULATION OR ABLATION

(71) Applicant: BOSTON SCIENTIFIC MEDICAL DEVICE LIMITED, Galway (IE)

(72) Inventors: Wentian ZHANG, Shanghai (CN); Xing XIA, Nanjing, Jiangsu (CN); Yinghua WANG, Shanghai (CN); Mingfeng XIE, Shanghai (CN); Cheng ZHANG, Shanghai (CN)

(73) Assignee: BOSTON SCIENTIFIC MEDICAL DEVICE LIMITED, Galway (IE)

(21) Appl. No.: 19/358,680

(22) Filed: Oct. 15, 2025

(30) Foreign Application Priority Data
Oct. 16, 2024 (CN) 202411448215.X

(51) Int. Cl. A61B 18/00; A61B 5/00; A61B 5/296; A61B 5/395

(52) U.S. Cl. CPC A61B 18/00; A61B 5/296; A61B 5/395; A61B 5/7228; A61B 2018/00178; A61B 2018/00351; A61B 2018/00577; A61B 2018/00613; A61B 2018/00642; A61B 2018/00666; A61B 2018/00708; A61B 2018/00726; A61B 2018/00839; A61B 2018/00982; A61B 2560/0468; A61B 2562/0219; A61B 2562/04; A61B 2562/227

(57) ABSTRACT

The present disclosure provides a medical system for performing pulse field ablation medical procedures. The medical system includes an ablation device configured to generate electrical fields that cause electroporation of tissue of a subject, a sensor array in electrical communication with the ablation device, and a control unit in electrical communication with the ablation device and the sensor array. The ablation device includes at least one set of electrodes. The sensor array includes at least one surface electromyography (sEMG) sensor and at least one inertial measurement unit (IMU) sensor. The control unit configured to generate a pulse waveform, transmit the pulse waveform to the ablation device, determine a muscle contraction level based on signal data from the ablation device and sensor data from the sensor array, and to determine an sEMG threshold based on the sensor data and muscle contraction level.

### CROSS-REFERENCE TO RELATED APPLICATIONS

This application claims the benefit of priority under 35 U.S.C. § 119 from Chinese Application No. 202411448215.X, filed Oct. 16, 2024, which is incorporated by reference herein in its entirety.

### TECHNICAL FIELD

Aspects of this disclosure generally relate to medical devices, systems, and related methods. Specifically, embodiments of the disclosure relate to medical devices, systems and methods for stimulation or ablation, for example, of tissue.

### BACKGROUND

Applying high voltage electrical pulses to tissue may generate electric fields in tissue and create a local region of ablated tissue by irreversible electroporation. Assessing the overall performance of the pulsed field ablation technology may be difficult, for example due to muscle contractions caused by pulsed field ablation. For example, the muscle contractions caused by pulsed field ablation may lead to an adverse event during the medical procedure, including electrode displacement, partial tissue ablation, or inadvertent damage to adjacent tissue. It would be useful to improve medical devices, systems, and related methods for ablation medical procedures.

### SUMMARY

Examples of the disclosure relate to, among other things, medical devices, systems, and related methods for pulsed electric field ablation of tissue.

According to one example, a medical system may include an ablation device including a set of electrodes configured to generate electrical fields. The electrical fields may cause electroporation of tissue of a subject contacting the ablation device. The medical system may include a sensor array in electrical communication with the set of electrodes of the ablation device, in which the sensor array may include at least one surface electromyography (sEMG) sensor and at least one inertial measurement unit (IMU) sensor. The medical system may include a control unit in electrical communication with the ablation device and the sensor array. The control unit may be configured to generate a pulse waveform; transmit the pulse waveform to the set of electrodes of the ablation device; determine a muscle contraction level based on signal data from the ablation device and sensor data from the sensor array; and determine an sEMG threshold based on sensor data from the sensor array and the muscle contraction level.

Any medical device or medical system described herein may include any of the following features. The ablation device may include a catheter having an insertion portion, in which the set of electrodes may be positioned at a distal end of the insertion portion. The catheter may include at least one of a balloon, a coil, a needle, or a basket. The tissue of the subject that contacts the catheter may include cardiac tissue. At least one sEMG sensor of the sensor array may include a plurality of sEMG sensors, and at least one IMU sensor of the sensor array may include a plurality of IMU sensors. The sensor array may include a housing configured to affix each sensor on epithelium tissue of the subject. The medical system may include a pad, and the pad may be configured to be coupled to epithelium tissue of the subject. The medical system may include at least one electrical wire, and the at least one electrical wire may be configured to provide wired electrical communication between at least one sensor of the sensor array and the control unit. The medical system may include at least one controller configured to provide wireless electrical communication between at least one sensor of the sensor array and the control unit.

The control unit may be configured to modulate at least one parameter of the pulse waveform based on at least one of sensor data from the sensor array and signal data from the ablation device. The control unit may be configured to modulate a duty cycle of the pulse waveform. The control unit may be configured to receive at least one assessment score corresponding to user evaluation of muscle contraction of the subject in response to the pulse waveform, and configured to modulate the pulse waveform based on the at least one assessment score. The control unit may be configured to determine at least one sEMG threshold based on the muscle contraction level. The control unit may be configured to generate a report based on the muscle contraction level and the sEMG threshold. The control unit may be configured to shutdown energy transmission to the ablation device based on the sEMG threshold and at least one measurement from the at least one sEMG sensor of the sensor array. The control unit may be configured to execute a machine learning model on historical signal data, and to determine the muscle contraction level based on an output from the machine learning model.

According to another example, a method for performing a medical procedure may include generating a pulse waveform with a control unit; transmitting the pulse waveform from the control unit to a set of electrodes of an ablation device in electrical communication with the control unit; determining a muscle contraction level based on signal data from the ablation device and sensor data from a sensor array in electrical communication with the control unit; or determining an sEMG threshold based on sensor data from the sensor array and the muscle contraction level.

Any method described herein may include any of the following features. The method may include inserting the ablation device into a body cavity of a subject, and positioning the ablation device in contact with or adjacent to tissue of the subject. The method may include measuring electrical signals from the set of electrodes of the ablation device. The method may include affixing the sensor array on epithelium tissue of the subject. The method may include measuring electrical signals via the sensor array, wherein the sensor array includes at least one sEMG sensor and at least one IMU sensor. The method may include modulating at least one parameter of the pulse waveform based on at least one of sensor data from the sensor array and signal data from the ablation device. Modulating at least one parameter of the pulse waveform may include modulating a duty cycle of the pulse waveform.

The method may include receiving at least one assessment score via the control unit, and the at least one assessment score may correspond to user evaluation of muscle contraction of the subject in response to the pulse waveform. The method may include modulating at least one parameter of the pulse waveform based on the at least one assessment score. The method may include generating a report based on the muscle contraction level and the sEMG threshold. The method may include shutting down energy transmission to the ablation device based on the sEMG threshold and at least one measurement from the at least one sEMG sensor of the sensor array. The method may include executing a machine learning model on sensor signal data, and determining the muscle contraction level based on an output from the machine learning model. The method may include training the machine learning model on historical sensor signals and muscle contraction levels.

According to yet another example, a medical system for performing a medical procedure may include an ablation device, a sensor array, and a control unit in electrical communication with the ablation device and the sensor array. The control unit may include at least one processor, and at least one memory to store program instructions executed by the at least one processor to execute steps for performing the medical procedure. The steps may include generating a pulse waveform with the control unit; transmitting the pulse waveform from the control unit to at least one set of electrodes of the ablation device; determining a muscle contraction level based on signal data from the ablation device and sensor data from the sensor array; and determining an sEMG threshold based on sensor data from the sensor array and the muscle contraction level. The method may also include modulating the pulse waveform with the control unit based on signal data from the ablation device and sensor data from the sensor array.

Any of the examples described herein may have any of these features in any combination.

### BRIEF DESCRIPTION OF THE DRAWINGS

The accompanying drawings, which are incorporated in and constitute a part of this disclosure, illustrate exemplary aspects of the disclosure and together with the description, serve to explain the principles of the disclosure.

FIG. 1 depicts a medical system, according to aspects of the disclosure.

FIG. 2 depicts another medical system, according to aspects of the disclosure.

FIGS. 3 and 4 depict methods, according to aspects of the disclosure.

FIG. 5 depicts an example computing device, according to aspects of the disclosure.

### DETAILED DESCRIPTION

Examples of the disclosure include devices, systems and related methods for stimulation or ablation, for example, of tissue of a subject via selective and rapid application of electric pulse waveforms, resulting in irreversible electroporation in tissue of the subject.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. As used herein, the terms “comprises,” “comprising,” “having,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value. As used herein, the phrase “based on” is understood to be equivalent to the phrase “based at least on,” unless indicated otherwise. The term “or” is used disjunctively, such that “at least one of A or B” includes, (A), (B), (A and A), (A and B), (B and B), etc.

Reference will now be made in detail to examples of the disclosure described above and illustrated in the accompanying drawings. Wherever possible, the same reference numbers will be used throughout the drawings to refer to the same or like parts.

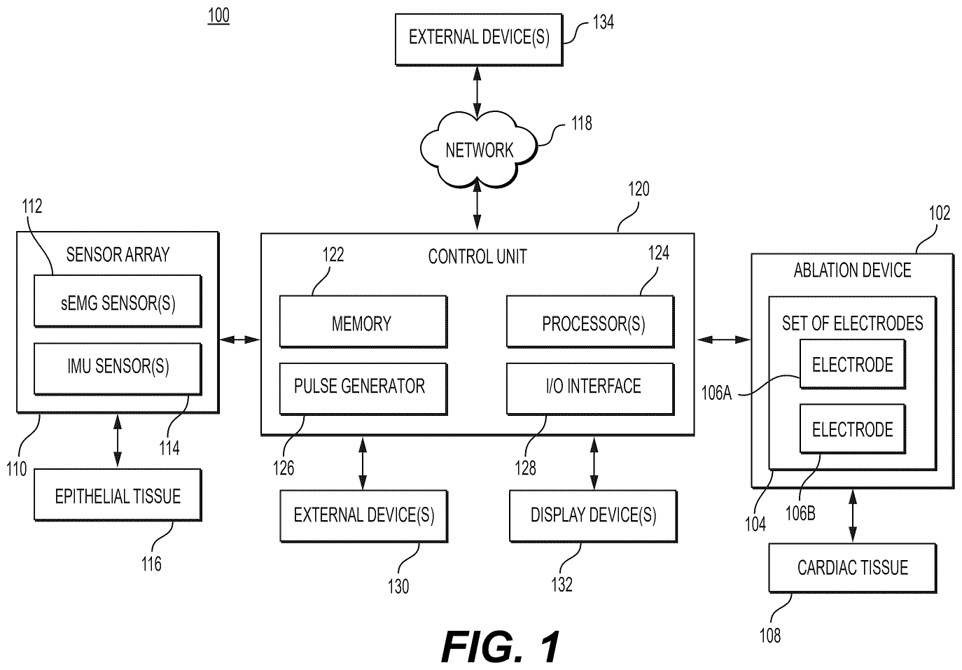

FIG. 1 depicts an exemplary medical system 100 according to aspects of the disclosure. Medical system 100 may include an ablation device 102, a sensor array 110, and a control unit 120. As discussed herein, medical system 100 may be configured to monitor cardiac activity (e.g., ectopic cardiac activity) of a patient in connection with tissue ablation via selective or rapid application of electric pulse waveforms at a target treatment site, or to monitor electrical signals from a muscular response to pulse waveforms. In the example shown, ablation device 102 may be coupled to control unit 120, and ablation device 102 may be configured to ablate cardiac tissue via pulse waveforms generated by control unit 120 in electrical communication with ablation device 102. Additionally, sensor array 110 may be affixed to epithelial tissue to measure electrical signals corresponding with muscular response of the patient. Control unit 120 may be configured to analyze sensor signal data (e.g., received from sensor array 110), for example to modulate subsequent pulse waveforms or stop delivery of ablation energy to ablation device 102.

In some aspects, ablation device 102 may be delivered through various insertion devices or delivery systems to the target site. For example, although not shown, ablation device 102 may be delivered through a working channel or other lumen of an endoscope, duodenoscope, gastroscope, colonoscope, ureteroscope, bronchoscope, or various other insertion devices or delivery systems to the target site. The medical system 100 may include a handle (not shown) having one or more ports that are configured to receive or control one or more medical instruments therein or one or more actuators. For example, the handle of medical system 100 may be configured for insertion of ablation device 102 into a body lumen of the patient, for example, to ablate cardiac tissue at the target site.

Ablation device 102 may include a catheter or shaft with one or more end effectors at the distal end or distal portion. For example, ablation device 102 may include at least one of a balloon, a coil, a needle, a basket, or the like at the distal end or distal portion. Ablation device 102 may include an insertion portion having a distal portion configured to be inserted into and navigated through a body lumen of the patient to the target site during a medical procedure, such that ablation device 102 is positioned proximate the target site. In some examples, ablation device 102 may be introduced into an endocardial space. For example, ablation device 102 may be introduced into the endocardial space of a left atrium through an atrial septum via a trans-septal puncture. Ablation device 102 may be a single use device that is discarded or disposed upon disconnection from control unit 120 (e.g., at an end of the medical procedure). Alternatively, one or more portions of medical system 100 may be reusable, for example, in more than one medical procedure. As discussed herein, ablation device 102 may be inserted into the patient, such that the distal portion contacts tissue (e.g., cardiac tissue 108) at the target site to perform one or more diagnostic or non-invasive medical procedures.

Ablation device 102 may include one or more electrodes configured to deliver pulse electric field energy (e.g., pulse waveform). Ablation device 102 may include a set of electrodes 104 configured to generate electrical fields, which may cause irreversible electroporation of proximate tissue (e.g., cardiac tissue 108 of the patient). For example, set of electrodes 104 may include independently energizable electrodes, such as an electrode 106A and an electrode 106B positioned in a distal tip of an insertion portion of ablation device 102. Electrodes 106A, 106B may include insulated electrical leads configured to sustain an electrical potential difference across a thickness without dielectric breakdown (e.g., from about 2,000 V to about 5,000 V). In some examples, set of electrodes 104 may be grouped into one or more anode-cathode subsets, such as a subset including one anode and one cathode, a subset including two anodes and one cathode, a subset including two cathodes and one anode, or the like. In the example shown in FIG. 1, set of electrodes 104 includes electrode 106A and electrode 106B. However, it should be understood that ablation device 102 may include any suitable number of electrodes, or sets of electrodes. For example, ablation device 102 may include a plurality of electrodes grouped into two, three, four, five, six, seven, eight or more sets of electrodes, which collectively or independently deliver ablation pulse waveforms to tissue proximate ablation device 102. In some examples, one or several external pad(s) attached on the patient skin may serve as the cathode electrode(s), which may help to improve ablation efficiency, for example, by working with the anode electrode(s) inserted into the body cavity.

According to some aspects, medical system 100 may be configured to collect or measure electrical signals corresponding to tissue activity (e.g., activity of cardiac tissue 108) in response to application of pulse waveforms from control unit 120 to ablation device 102. For example, ablation device 102 may be configured to transmit electrical signals from set of electrodes 104, for example, to control unit 120 or various other external computing device(s) discussed herein. Medical system 100 may include a sensor array 110 having one or more sensors. Sensor array 110 may be configured to measure one or more muscle responses or electrical activity of the patient (e.g., during the medical procedure in real-time or designated intervals). Sensor array 110 may include one or more surface electromyography (sEMG) sensors 112 or one or more inertial measurement unit (IMU) sensors 114. As such, sensor array 110 may measure one or more muscle responses, for example, in response to electrical stimulation of cardiac tissue 108 via ablation device 102.

Medical system 100 may include electrical components that support or otherwise provide electrical communication between control unit 120 and sensor array 110. For example, one or more electrical wires configured to provide wired electrical communication, directly or indirectly, may extend between control unit 120 and at least one of sEMG sensor(s) 112 or IMU sensor(s) 114 of sensor array 110. This electrical communication may support data transmission of sensor signal data from sensor array 110 to control unit 120. For example, each sEMG sensor(s) 112 or each IMU sensor(s) 114 may be directly coupled to control unit 120 via respective electrical wires, such that control unit 120 may process or analyze data received from independent sensors of sensor array 110.

In some aspects, sensor array 110 may include a housing secured to one or more sEMG sensor(s) 112 or IMU sensor(s) 114. The housing may be configured to affix or otherwise couple sEMG sensor(s) 112 or IMU sensor(s) 114 on epithelial tissue 116 of the patient during the medical procedure. In some examples, the housing may include one or more clothing articles affixed to sEMG sensor(s) 112 or IMU sensor(s) 114. For example, the housing may include a belt, wristband, vest, shirt, pants, or various other clothing articles secured to sEMG sensor(s) 112 or IMU sensor(s) 114. The housing may include one or more elastic materials for compressing or otherwise urging sensor array 110 against or toward epithelial tissue 116, for example, to help maintain contact between the patient and sensor array 110 while measuring one or more muscular responses to application of pulse waveforms.

In alternative implementations, the housing may include one or more input ports that support electrical communication between sensor array 110 and control unit 120. For example, control unit 120 may in electrical communication with one or more ports of the housing, such that each of sEMG sensor(s) 112 and IMU sensor(s) 114 are indirectly coupled to control unit 120 via the port(s) of the housing that houses sensor array 110 therein. The housing may include electrical wiring or circuits that support adding or removing one or more other sensors to sensor array 110.

In some aspects, although not shown, medical system 100 may optionally include a pad affixed or otherwise coupled to epithelial tissue 116, which may help facilitate measuring sensor signal data via sensor array 110 during the medical procedure. For example, the pad of medical system 100 may be useful to abrade epithelial tissue 116 (e.g., to remove dead skin cells of the patient), for example, to help improve electrical connections between sensor array 110 and epithelial tissue 116.

Control unit 120 may be a computing system, a controller, a computing device, or other similar standalone processing unit separate from and selectively connectable to ablation device 102. Control unit 120 may include a memory 122 and one or more processors 124. Memory 122 may store instructions to be executed by processor(s) 124 to cause control unit 120 to perform corresponding operations. Memory 122 may include a computer readable storage medium for storing data, for example, electrical signal data from ablation device 102 or sensor data from sensor array 110. Processor(s) 124 may be or include a central processing unit (CPU), graphics processing unit (GPU), tensor processing unit (TPU), field-programmable gate array (FPGA), or the like.

Control unit 120 may communicate with one or more devices in or via a network 118 via input/output (I/O) interface 128. I/O interface 128 may support electrical communication with ablation device 102 or sensor array 110. In some examples, medical system 100 may include an umbilicus configured to be coupled with I/O interface 128. The umbilicus may provide power transmission to ablation device 102 or data transmission to and from ablation device 102, such as electrical signal data from electrodes 104 corresponding to ectopic cardiac activity of the patient. In other implementations, data transmission may include auxiliary data from one or more auxiliary sensors, such as pressure sensors or optical sensors.

Control unit 120 may include a pulse generator 126 configured to generate ablation pulse waveforms for delivery to ablation device 102. For example, pulse generator 126 may include an electric pulse waveform generator to generate and deliver pulse waveforms to electrodes 104, in turn treating tissue adjacent to ablation device 102. For example, the pulse waveforms delivered to electrodes 104 may cause irreversible electroporation of tissue (e.g., cardiac tissue) adjacent to ablation device 102. Pulse generator 126 may generate and deliver various types of signals including, but not limited to, monophasic electric pulses and biphasic electric pulses. Control unit 120 may be configured to modulate one or more parameters (e.g., amplitude, duty cycle, width, timing, etc.) of pulse waveforms output from pulse generator 126. Medical system 100 may include electrical components that support or otherwise provide electrical communication between control unit 120 and set of electrodes 104. For example, one or more electrical wires configured to provide wired electrical communication, directly or indirectly, may extend from control unit 120 and to set of electrodes 104.

According to some aspects, control unit 120 may be configured to modulate pulse waveform parameters based on analysis of electrical signal data received from ablation device 102 or sensor signal data received from sensor array 110. Control unit 120 may be configured to iteratively modulate pulse waveform parameter(s). In some examples, control unit 120 may use pulse-width modulation (PWM) to iteratively adjust duty cycle or use pulse-amplitude modulation (PAM) to iteratively adjust amplitude. Control unit 120 may be configured to log or store data associated with pulse waveforms, for example logging waveform transmission data to a database stored in memory 122 over one or more time periods of pulse waveform transmission between pulse generator 126 and ablation device 102.

In some aspects, medical system 100 may include one or more external devices 130 coupled to control unit 120 (e.g., via I/O interface 128). In some examples, external device(s) 130 may be used to manipulate one or more user interfaces generated by control unit 120, such as a graphical user interface (GUI) shown on a display device 132 of medical system 100. It should be understood that external device(s) 130 may be separate from display device 132. Control unit 120 may be configured to process or display (or transmit for display) data associated with ablation device 102, sensor array 110, or control unit 120, for example, via display device 132. For example, during the medical procedure, display device 132 may display electrical signal data received from ablation device 102 or may display sensor signal data received from sensor array 110. In some examples, control unit 120 may be configured to display (e.g., via display device(s) 132) parameters associated with pulse waveforms transmitted to electrodes 104 of ablation device 102 (e.g., over a period of time). Similarly, control unit 120 may be configured to display (e.g., via display device(s) 132) sensor signal data associated with muscular response measured by sensor array 110 (e.g., in response to pulse waveforms to ablation device 102 over the period of time). In some examples, a user may manipulate or otherwise input one or more selections via the interface to select, display, or otherwise interact with data or other information provided via display device 132 (e.g., during medical procedures).

According to some aspects, control unit 120 may be configured to receive one or more assessment scores correspond to medical profession evaluation(s) of muscle contraction in response to application of electric pulse waveforms (e.g., to cardiac tissue of the patient via ablation device 102). For example, the assessment score(s) may correspond to evaluation by the medical professional including visual confirmation of muscle contraction, or lack thereof, in response to delivering the pulse waveform from pulse generator 126 to set of electrodes 104 proximate cardiac tissue of the patient. Control unit 120 may be configured to prompt the medical professional(s) display device 132 for assessment input during a time period of pulse waveform delivery to the patient via ablation device 102 during the medical procedure.

As discussed herein, control unit 120 may be configured to modulate pulse waveforms based on the sEMG data received from sensor array 110, for example, to adjust (e.g., iteratively adjust) at least one parameter of pulse waveforms output from pulse generator 126 based on analysis of assessment score(s). For instance, control unit 120 may be configured to adjust (e.g., iteratively adjust) a duty cycle of the pulse waveform based on the sEMG data, and to deliver the pulse waveform having the adjusted duty cycle to electrodes 104 of ablation device 102. Pulse waveform modulation may continue with iterative adjustments to pulse waveform parameter(s), for example, until specified criteria are met or otherwise indicate energy delivery from control unit 120 and to ablation device 102 should not continue. For example, control unit 120 may adjust (e.g., increase) one or more pulse waveform parameter(s) until either the upper limit of the parameter(s) is met or until muscle contraction from ablation energy delivery interferes with the medical procedure. In one example, if the assessment score associated with the sEMG data indicates no muscle contraction or muscle contraction level is safe in the patient, control unit 120 may continue increasing ablation energy delivery until a subsequent assessment score associated with the sEMG data is received, which indicates muscle contraction level is reach to the risk threshold at the corresponding pulse waveform parameter(s).

Control unit 120 may be configured to report a muscle contraction level based on an input data set including, but not limited to, parameter(s) of pulse waveforms transmitted by control unit 120, electrical signal data measured by ablation device 102, sensor signal data measured by sensor array 110, or historical signal dataset. Control unit 120 may be configured to execute instructions accessing the input data set (e.g., stored in memory 122 or external database via network 118), and analyzing the dataset (e.g., via processor(s) 124) to determine the muscle contraction level under the respective pulse field ablation (PFA) dose. The “PFA dose” including the pulse waveform transmitted by control unit 120 having specified parameters over a specified period of time. The medical device 100 may define a lower boundary for pulse waveform parameters (e.g., lowest PFA dose) or define an upper boundary for pulse waveform parameters (e.g., highest PFA dose).

According to some aspects, control unit 120 may be configured to determine an sEMG threshold based on the sEMG data acquired by sensor array 110 and on the muscle contraction assessment result(s) provided by medical professional(s). The sEMG threshold may be determined from a muscle contraction threshold corresponding with the safest muscle contraction level and lowest PFA dose for the subject. The muscle contraction threshold may be determined by the medical professional(s) observation of body movement (e.g., of the patient's body in response to the PFA dose) and the medical professional(s) assessment of risk level during the medical procedure (e.g., risk that body movement interferes with ablation device 102, sensor array 110 or other risk of injury to the subject).

Prior to commencing the medical procedure, the sEMG sensor(s) 112 and IMU sensor(s) 114 may be affixed to the patient, such as affixed to upper limbs, lower limbs, torso, etc. of the patient. Control unit 120 may commence pulse waveform delivery from the lower boundary of PFA dose (e.g., lowest PFA amplitude, narrowest pulse width, and smallest number of pulses), while collecting sensor signal data via sEMG sensor(s) 112 and IMU sensor(s) 114 (e.g., in response to PFA dose). The medical professional(s) evaluate and record the impact of the body movement caused by the muscle contraction under the PFA dose, for example which may be input to control unit 120 for analyzing the medical professional(s) evaluation. Pulse waveform parameter(s) may be iteratively adjusted with corresponding documentation of the medical professional's assessments at adjusted PFA doses having the adjusted waveform parameter(s). This iterative process may continue, for example, (1) until the patient's body movement caused by ablation-induced muscle contraction interferes with the medical procedure, (2) the medical professional(s) stop the test, or (3) the upper boundary of parameters is reached.

In some examples, a first assessment score corresponding with sEMG data at a first time can be viewed as first sEMG threshold (T₁), a second assessment score corresponding with sEMG data at a second time can be viewed as second sEMG threshold (T₁), etc., until an n^{th} assessment score corresponding with sEMG data at an n^{th} time can be viewed as n^{th} sEMG threshold (Tₙ). A final sEMG threshold may be determined, for example, by averaging each sEMG threshold (e.g., T, T₂, Tₙ, etc.).

In some examples, control unit 120 is configured to execute a machine learning model (“ML model”) trained on historical dataset, and to determine/refine muscle contraction threshold based on an output from the ML model. The historical dataset may include historical data of one or more of sEMG sensor signals, IMU sensor signals, PFA dose, cardiac tissue signal data, sEMG thresholds and the muscle contraction score associated with the sEMG thresholds, body mass index (BMI) of the patient, etc. The ML model may include a neural network trained via the historical signal data, and the neural network may be configured to output predictions for the muscle contraction level. During the medical procedure, the ML model may be configured to receive sensor data from sensor array 110 as an input, and to determine the muscle contraction level based on sensor data and PFA dose and output the sEMG threshold prediction. In some examples, the ML model may be a semi-supervised learning model that receives at least one assessment from the user during the medical procedure, for example to predict and output the muscle contraction level. In some examples, the ML model may be a reinforcement learning model that optimizes one or more parameters for the PFA doses(s) based on the historical dataset. In some examples, medical system 100 is configured to continue training the ML model for improving performance based on additional data, for example to improve prediction accuracy of muscle contraction level at various PFA doses.

In some aspects, control unit 120 may compare measurements from sEMG sensor(s) 112 of sensor array 110 with the sEMG threshold. Control unit 120 may be configured to perform one or more operations based on this comparison. For example, control unit 120 may shutdown, stop, reduce, or otherwise adjust energy output to ablation device 102 based on the sEMG threshold and measurement(s) received from sEMG sensor(s) 112. For example, if the measurement exceeds the sEMG threshold, then control unit 120 may be configured to cease energy output to ablation device 102. If the measurement does not exceed the sEMG threshold, then control unit 120 may continue delivering energy to ablation device 102 and continue receiving sensor signals via sensor array 110 (e.g., until sensor measurements reach or exceed the sEMG threshold). Typically, sEMG sensor signal precedes body movement of the patient in a range from about 30 milliseconds to about 150 milliseconds. Control unit 120 therefore may help safely perform the operation(s) based on the comparison (e.g., shutdown energy delivery to ablation device 102) without damaging tissue of or otherwise injuring the patient during the medical procedure (e.g., due to high voltage energy delivery).

According to some aspects, control unit 120 may be configured to generate a report based on sEMG data acquired. In some examples, the report may include measurements from sensor array 110 or ablation device 102. In some examples, the report may include information about the muscle contraction level assessment score. Control unit 120 may provide the report to one or more specified users, for example by executing program instructions to electronically transmit generated reports to the specified user(s) via e-mail or the like. Control unit 120 may be further configured to store generated reports in a local storage medium, remote storage medium, or combination thereof.

Although only one control unit 120 is shown in FIG. 1, in some examples, medical system 100 may include more than one control units 120 that are similar to, or the same as, control unit 120. In these aspects, ablation device 102 (or another medical device) may also be connected to each of control unit(s) 120. To provide an illustrative example, within a medical facility, multiple procedural suites may each include one control unit 120 that remains located in that suite, and ablation device 102 may be selectively coupled or uncoupled to the control unit 120 in the particular suite.

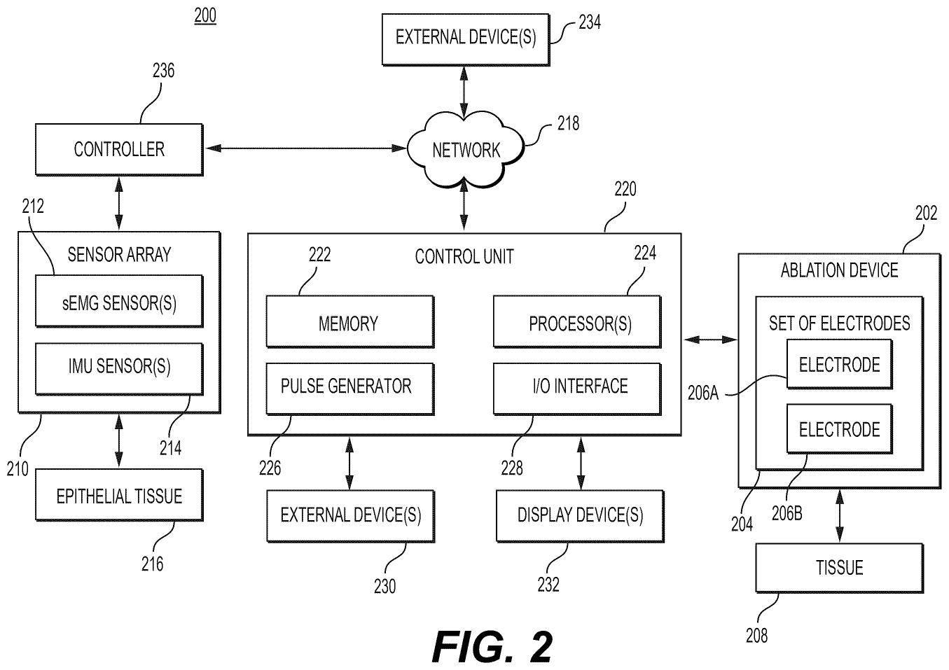

FIG. 2 illustrates another exemplary medical system 200 including an ablation device 202, a sensor array 210, and a control unit 220. As discussed above, control unit 220 is in electrical communication with ablation device 202 and sensor array 210. Unless otherwise specified, medical system 200 may include one or more aspects of medical system 100 (FIG. 1), the details of which are omitted for brevity.

As shown, ablation device 202 includes a set of electrodes 204 having, at least, an electrode 206A and an electrode 206B. Ablation device 202 may deliver ablation pulse waveform energy to tissue 208 of a patient adjacent to set of electrodes 204, such as cardiac tissue. It should be understood that tissue 208 is not limited to cardiac tissue, such that ablation device 202 may deliver ablation pulse waveform energy to other types of tissue, for example including but not limited to liver tissue, prostate tissue, and pulmonary tissue. Sensor array 210 includes one or more sEMG sensors 212 and one or more IMU sensors 214. Sensor array 210 may collect or measure sensor signal data while affixed to epithelial tissue 216 of the patient. Control unit 220 includes a memory 222, one or more processors 224, a pulse generator 226 configured to generate ablation pulse waveforms, and an I/O interface 228. Control unit 220 may be in electrical communication with external device(s) 230 or display device(s) 232. Control unit 220 may be directly coupled to ablation device 202 (e.g., via an umbilicus or the like). Control unit 220 may be configured to communicate with one or more external devices 234 via a network 218.

As shown and discussed herein, medical system 200 may include one or more wireless controllers configured to provide wireless electrical communication between one or more sensors of sensor array 210 and control unit 220. For example, medical system 200 may include a controller 236 in electrical communication with sensor array 210 and control unit 220. Controller 236 may be configured to receive sensor signals from one or more sensors in sensor array 210, and to wirelessly transmit sensor signal data from sensor array 210 and to control unit 220. For example, controller 236 may wirelessly transmit sensor signal data corresponding to measurements from sEMG sensor(s) 212 or from IMU sensor(s) 214. Controller 236 may directly or indirectly couple to sEMG sensor(s) 212 or IMU sensor(s) 214, for example, via electrical components that support electrical communication between controller 236 and sensor array 210. In some examples, a plurality of electrical wires may collectively be configured to support or provide electrical communication between controller 236 with each IMU sensor(s) 212 or each sEMG sensor(s) 214. In other examples, sEMG sensor(s) 212 or IMU sensor(s) 214 may include a transmitter in electrical communication with controller 236, for example to support wireless communication for transmission of sensor signal data from sEMG sensor(s) 212 or IMU sensor(s) 214 to controller 236. Controller 236 may wirelessly transmit sensor signal data directly or indirectly to control unit 220, for example, via a local area network (LAN) or wide area network (WAN) in electrical communication with controller 236, such as network 218. Although not shown, controller 236 may include one or more components or auxiliary devices configured to perform computing operation(s), such as memory, processor(s), I/O interface, etc.

In some implementations, controller 236 may execute program instructions to process raw sensor signals received from sensor array 210, for example, to filter noise from raw sensor signals to improve a signal-to-noise ratio of resulting sensor signal data (e.g., prior to transmission of sensor signal data). In some implementations, controller 236 may execute program instructions to transmit sensor signal data to one or more external device(s) 234 via network 218, such as external database(s) for logging sensor signal data over one or more time periods.

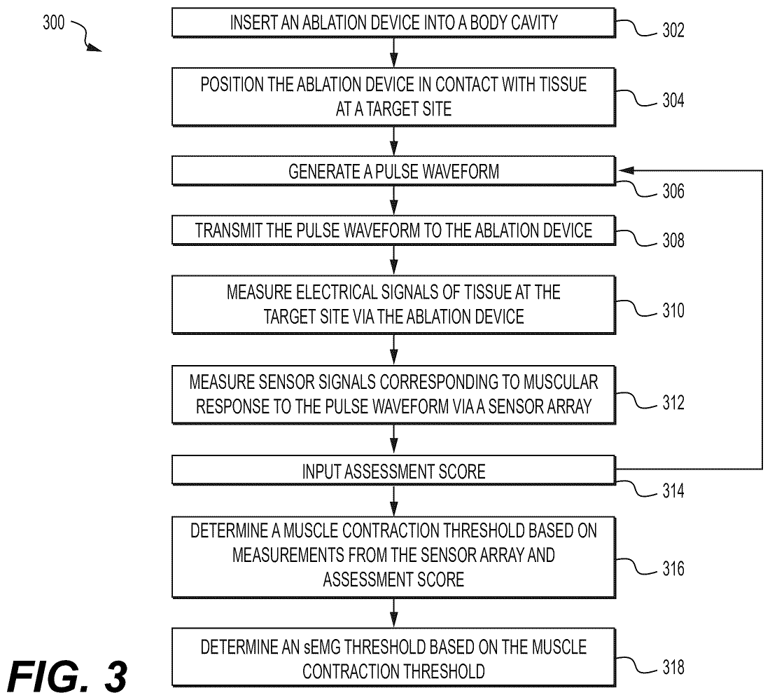

FIG. 3 shows a method 300 for ablating tissue during a medical procedure, according to aspects of the disclosure. A step 302 of method 300 includes inserting an ablation device into a body cavity. For example, inserting ablation device 102 of medical system 100 (FIG. 1) into a body lumen of a patient may include inserting a medical device into the body lumen and inserting ablation device 102 through a lumen or channel of the medical device. A step 304 of method 300 includes positioning the ablation device in contact with or adjacent to tissue at a target site. For example, step 304 may include inserting a distal portion of ablation device 102 having set of electrodes 104 (FIG. 1) in contact with or adjacent cardiac tissue at the target site within the patient during the medical procedure. A step 306 of method 300 includes generating a pulse waveform. For example, step 306 may include generating ablation pulse waveforms with pulse generator 126 of control unit 120 (FIG. 1). A step 308 of method 300 includes transmitting the pulse waveform to ablation device. For example, transmitting ablation pulse waveforms may include transmitting ablation pulse waveforms from pulse generator 126 of control unit 120 to electrodes 104 of ablation device 102 (FIG. 1).

A step 310 of method 300 includes measuring electrical signals of tissue at the target site via the ablation device. For example, step 310 may include measuring ectopic cardiac activity of cardiac tissue at the target site via set of electrodes 104 in response to application of the pulse waveform. Step 310 may further include transmitting electrical signal data from the ablation device and to the control unit, for example, via the umbilicus supporting electrical communication between ablation device 102 and control unit 120 (FIG. 1). A step 312 of method 300 includes measuring sensor signals that correspond to a muscular response to the pulse waveform via a sensor array. For example, step 312 may include measuring sensor signals including sEMG signals via sEMG sensor(s) 112 or IMU signals via IMU sensor(s) 114. Step 312 may further include transmitting sensor signal data from the sensor array and to the control unit, for example, via electrical wires supporting electrical communication between control unit 120 and sensor array 110 (FIG. 1), or wireless transmission of sensor signal data to control unit 220 via controller 236 (FIG. 2.).

A step 314 of method 300 includes inputting an assessment score. For example, step 314 may include the medical professional submitting assessment score(s) that corresponds to observation of muscular response in the patient to ablation pulse waveforms delivery. A step 316 of method 300 includes determining a muscle contraction threshold based on the assessment score. For example, step 316 may include determining muscle contraction threshold based on assessment score(s) from the medical professional(s), or electrical signal data from ablation device 102, or BMI of the patient to determine the muscle contraction threshold.

A step 318 of method 300 includes determining an sEMG threshold based on the determined muscle contraction threshold and the sEMG data. For example, step 318 may include analyzing the muscle contraction threshold, sensor signal data received from sEMG sensor(s) 112, assessment score(s) from the medical professional, or historical data including historical sensor signal data to determine the sEMG threshold.

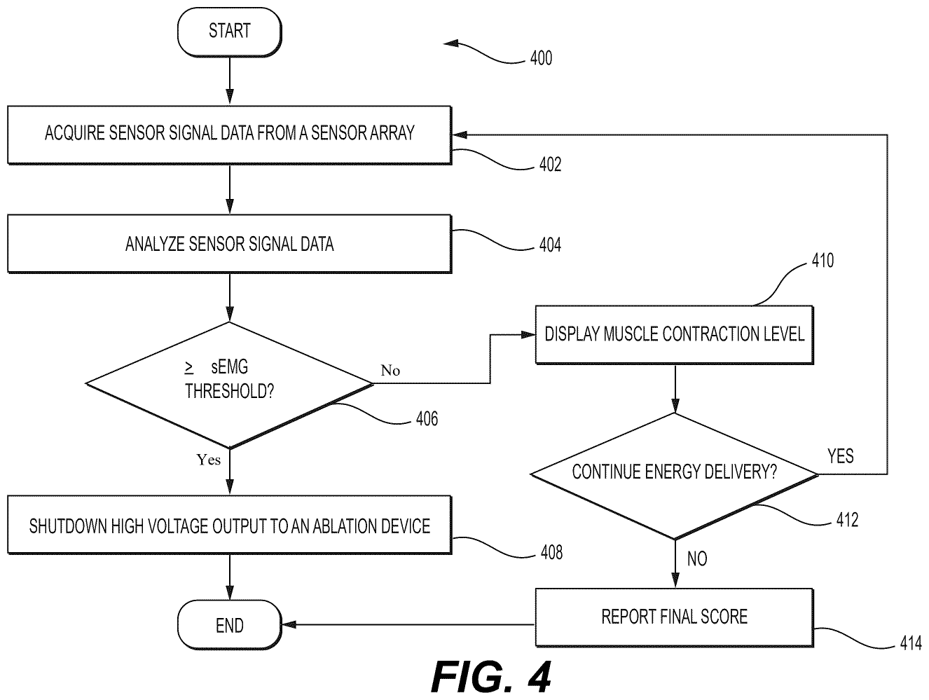

FIG. 4 shows a flow chart of a method 400 for ablating tissue during a medical procedure, according to aspects of the disclosure.

A step 402 of method 400 includes acquiring sensor data from a sensor array. For example, step 402 may include acquiring sensor signal data may include sEMG measurements from sEMG sensor(s) 112 of sensor array 110 (FIG. 1) while affixed or otherwise coupled to epithelial tissue of the patient during the medical procedure. Sensor signal data may include sEMG measurements at various points during a time period. A step 404 of method 400 includes analyzing sensor signal data. For example, step 404 may include analyzing sensor signal data from sEMG sensor(s) 112 (FIG. 1) to determine at least one sEMG measurement for at least one instance over the time period in the medical procedure. In some examples, step 404 may include processing sensor signal data from sEMG sensor(s) 112, for example to improve signal-to-noise ratio and facilitate analysis of sensor signal data.

A step 406 of method 400 includes comparing an sEMG threshold with the sEMG measurement determined in Step 404. If the sEMG threshold is met or exceeded, then method 400 proceeds to a step 408 that includes stopping high voltage output to an ablation device. In some aspects, step 408 includes displaying the latest muscle contraction score or error notice. For example, control unit 120 may be configured to stop or otherwise change ablation energy output to ablation device 102 in response to determining that measurements from sEMG sensor(s) 112 exceed the sEMG threshold. In some examples, step 408 may include generating or displaying an error notice during the medical procedure. For example, control unit 120 may be configured to generate the error notice and to show, display, or otherwise indicate the error notice on display device 132 indicating sEMG threshold has been met or exceeded based on analysis of sensor signal data from sEMG sensor(s) 112.

Returning to step 406, if the sEMG threshold is not met or exceeded, then method 400 proceeds to a step 410 that includes displaying a muscle contraction level. For example, processing sensor signal data via control unit 120 and showing the determined muscle contraction level to the medical professional(s) via display device 132 (FIG. 1).

Method 400 proceeds to a step 412, which includes determining whether to continue energy delivery. For example, the medical professional may determine whether to continue energy delivery from control unit 120 to ablation device 102 based on the muscle contract level shown via display device 132 (FIG. 1). In some examples, medical system 100 may be configured to prompt the user to determine whether control unit 120 should continue energy delivery (e.g., to set of electrodes 104). If the user selects to continue energy delivery, then method 400 may return, for example, to continue energy delivery to ablation device 102 and in turn continue acquiring sensor signal data at step 402 (e.g., via sensor array 110). This loop or repetition of various steps of method 400 may continue indefinitely until the sEMG threshold is exceeded at step 406, or until the user elects to discontinue energy delivery at step 412 (or otherwise stops the medical procedure). If the user selects to discontinue energy delivery at step 412, then method 400 proceeds to a step 414 of method 400, which includes reporting a final score. For example, control unit 120 may be configured to report a summary of the medical procedure, including electrical signal data from ablation device 102, sensor signal data from sensor array 110, and modulation of pulse waveforms from control unit 120 over the course of the medical procedure. Step 414 may include generating the report via control unit 120, and delivering the generated report to one or more specified users (e.g., via e-mail or the like).

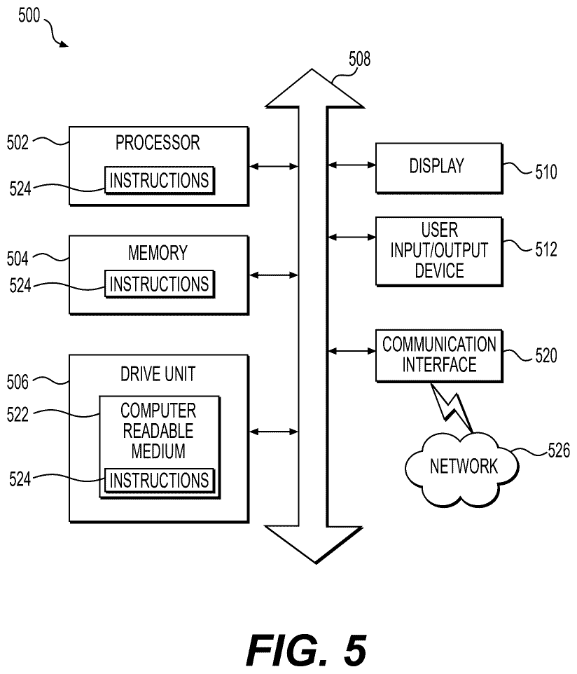

FIG. 5 depicts an example of a computer 500. FIG. 5 is a simplified functional block diagram of computer 500 that may be configured as a device for executing processes, steps, or operations depicted in, or described with respect to, FIGS. 1-4 and, according to exemplary embodiments of this disclosure. For example, computer 500 may be configured as one or more of medical system 100, control unit 120, or another external device 134 or component, according to exemplary embodiments of this disclosure. In various embodiments, any of the systems herein may be or include computer 500 including, e.g., a data communication interface 520 for packet data communication. Computer 500 may communicate with one or more other computers, for example, using an electronic network 526 (e.g., via data communication interface 520). Electronic network 526 may include a wired or wireless network, for example, similar to optional network 118 depicted in FIG. 1.

Computer 500 also may include a central processing unit (“CPU”), in the form of one or more processors 502, for executing program instructions 524. In some examples, processors 502 may be or include one or more field-programmable gate arrays (FPGAs), a graphics processing unit (“GPU”), a tensor processing unit (“TPU”), an application-specific integrated circuit (“ASIC”), or any combination thereof. Program instructions 524 may include at least instructions for performing usage monitoring (e.g., if computer 500 is or is included in medical system 100). Program instructions 524 for carrying out operations of the disclosure may include source code or object code written in one or more programming languages, such as object oriented programming languages (e.g., Python, C++, etc.) or procedural programming languages (e.g., C).

Computer 500 may include an internal communication bus 508. Computer 500 may also include a drive unit 506 (such as read-only memory (ROM), hard disk drive (HDD), solid-state disk drive (SDD), etc.) that may store data on a computer readable medium 522 (e.g., a non-transitory computer readable medium), although computer 500 may receive programming and data via network communications. Computer 500 may also have a memory 504 (such as random-access memory (RAM)) storing instructions 524 for executing techniques presented herein. It is noted, however, that in some aspects, instructions 524 may be stored temporarily or permanently within other modules of computer 500 (e.g., processor 502 or computer readable medium 522). Computer 500 also may include user input and output devices 512 or a display 510 to connect with input or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical system, comprising:
an ablation device including a set of electrodes configured to generate electrical fields that cause electroporation of tissue of a subject contacting the ablation device;
a sensor array in electrical communication with the set of electrodes of the ablation device, wherein the sensor array includes at least one surface electromyography (sEMG) sensor and at least one inertial measurement unit (IMU) sensor; and
a control unit in electrical communication with the ablation device and the sensor array, wherein the control unit is configured to:
generate a pulse waveform;
transmit the pulse waveform to the set of electrodes of the ablation device;
determine a muscle contraction level based on signal data from the ablation device and sensor data from the sensor array; and
determine an sEMG threshold based on sensor data from the sensor array and the muscle contraction level.

2. The medical system of claim 1, wherein the ablation device includes a catheter having an insertion portion, and wherein the set of electrodes are positioned at a distal end of the insertion portion.

3. The medical system of claim 2, wherein the catheter includes at least one of a balloon, a coil, a needle, or a basket, and wherein tissue of the subject contacting the catheter includes cardiac tissue.

4. The medical system of claim 1, wherein at least one sEMG sensor of the sensor array includes a plurality of sEMG sensors, and wherein at least one IMU sensor of the sensor array includes a plurality of IMU sensors.

5. The medical system of claim 1, wherein the sensor array includes a housing configured to affix each sensor on epithelium tissue of the subject.

6. The medical system of claim 1, further comprising at least one electrical wire configured to provide wired electrical communication between at least one sensor of the sensor array and the control unit.

7. The medical system of claim 1, further comprising at least one controller configured to provide wireless electrical communication between at least one sensor of the sensor array and the control unit.

8. The medical system of claim 1, wherein the control unit is configured to modulate at least one parameter of the pulse waveform based on at least one of sensor data from the sensor array and signal data from the ablation device.

9. The medical system of claim 8, wherein the control unit is configured to modulate a duty cycle of the pulse waveform.

10. The medical system of claim 9, wherein the control unit is configured to receive at least one assessment score corresponding to user evaluation of muscle contraction of the subject in response to the pulse waveform, and configured to modulate the pulse waveform based on the at least one assessment score.

11. The medical system of claim 1, wherein the control unit is configured to generate a report based on the muscle contraction level and the sEMG threshold.

12. The medical system of claim 1, wherein the control unit is configured to shutdown energy transmission to the ablation device based on the sEMG threshold and at least one measurement from the at least one sEMG sensor of the sensor array.

13. The medical system of claim 1, wherein the control unit is configured to execute a machine learning model on historical signal data, and to determine the muscle contraction level based on an output from the machine learning model.

14. The medical system of claim 13, wherein the control unit is configured to execute the machine learning model on at least one assessment score corresponding to user evaluation of muscle contraction of the subject in response to the pulse waveform.

15. The medical system of claim 1, further comprising a pad configured to be coupled to epithelium tissue of the subject.

16. A method for performing a medical procedure comprising:
generating a pulse waveform with a control unit;
transmitting the pulse waveform from the control unit to a set of electrodes of an ablation device in electrical communication with the control unit;
determining a muscle contraction level based on signal data from the ablation device and sensor data from a sensor array in electrical communication with the control unit; and
determining an sEMG threshold based on sensor data from the sensor array and the muscle contraction level.

17. The method of claim 16, further comprising:
inserting the ablation device into a body cavity of a subject;
positioning the ablation device in contact with or adjacent to tissue of the subject, and
measuring electrical signals from the set of electrodes of the ablation device.

18. The method of claim 17, further comprising:
affixing the sensor array on epithelium tissue of the subject; an
measuring electrical signals via the sensor array, wherein the sensor array includes at least one sEMG sensor and at least one IMU sensor.

19. The method of claim 17, further comprising:
receiving at least one assessment score via the control unit, wherein the at least one assessment score corresponds to user evaluation of muscle contraction of the subject in response to the pulse waveform;
modulating at least one parameter of the pulse waveform based on the at least one assessment score; and
generating a report based on the muscle contraction level and the sEMG threshold.

20. A medical system for performing a medical procedure, the medical system comprising:
an ablation device;
a sensor array; and
a control unit in electrical communication with the ablation device and the sensor array, wherein the control unit includes at least one processor, and at least one memory to store program instructions executed by the at least one processor to execute steps for performing the medical procedure, wherein the steps comprise:
generating a pulse waveform with the control unit;
transmitting the pulse waveform from the control unit to at least one set of electrodes of the ablation device;
determining a muscle contraction level based on signal data from the ablation device and sensor data from the sensor array; and
determining an sEMG threshold based on sensor data from the sensor array and the muscle contraction level.
